(19) United States
(12) Patent Application Publication
(10) Pub. No.: US 2010/0019169 A1
(43) Pub. Date: Jan. 28, 2010

(54) REMOTE LASER INTERROGATION OF THREAT CLOUDS AND SURFACES

(75) Inventor: Claire Hartmann-Thompson, Midland, MI (US)

Correspondence Address: TECHNOLOGY LAW, PLLC, 3595 N. SUNSET WAY, SANFORD, MI, 48657, US

(73) Assignee: Michigan Molecular Institute, Midland, MI (US)

(21) Appl. No.: 12/449,081

(22) PCT Filed: Jan. 25, 2008
(86) PCT No.: PCT/US08/00994

§ 371 Date: Jul. 22, 2009

Related U.S. Application Data
(60) Provisional application No. 60/897,958, filed on Jan. 29, 2007.

(51) Int. Cl. G01J 1/58; C07D 215/00; C07D 491/107; C07D 271/08

(52) U.S. Cl. 250/459.1; 546/157; 548/410; 548/125

(57) ABSTRACT

This invention concerns the remote detection of threat clouds and evaluation of their components. Also included is the remote detection and evaluation of contamination on surfaces or in air streams. To enable this detection, fluorophores that are attached to POSS are used with two-photon LIF imaging that provides enhanced background-free imaging even in the presence of scattering particles such as dust, sand and water droplets.

### FEDERALLY SPONSORED RESEARCH STATEMENT

This invention was made with Government support under W911SR-05-C-0026 awarded by US Army RDCOM ACQ. Center [DARPA] the Department of Defense. The Government has certain rights in this invention.

### BACKGROUND OF THE INVENTION

1. Field of the Invention

This invention concerns the remote detection of threat clouds and evaluation of their components. Also included is the detection obtained remotely and evaluation of contamination on surfaces.

2. Description of Related Art

Remote sensing techniques must function either: (1) by passive detection of waves emitted from remote objects; or (2) by actively sending a wave to a remote object and obtaining information by analyzing the waves returning from the object. In an early report by Ludwig, C. B. et al., [Electron. Res. Center, General Dynamics Corp., NASA Contract Report (1969); NASA CR-1380] on the topic of remote detection of air pollutants from space, they correctly foresaw that the major technical obstacles in remote detection would be: (a) scattering caused by solid particles or droplets; and (b) measurement of low energy signals against a constantly changing background of temperature and radiation levels.

One method to try to improve this technique is to use molecules that emit light. POSS molecules are stoichiometrically well-defined cage compounds prepared by the hydrolysis and condensation of trifunctional silanes of the form RSiX₃ [see, for example, Scott, D., J. Am. Chem. Soc. 68, 356 (1946) and Varonkov, M. G. et al., Topics Curr, Chem. 102, 199-236 (1982)]. These condensation reactions can generate products ranging from small molecules, oligomers, and clusters to resins of highly complex structure. Which products are obtained from these condensation reactions is highly dependent upon silane and water concentration, pH, temperature, solubility and catalyst [see Brinker, C. J. et al., Sol-Gel Sci.: The Physics and Chemistry of Sol-Gel Processing, Academic Press, San Diego (1990)]. Some derivatized POSS molecules have been made that carry light-emitting lumophores for light emitting applications, preferably white light emitters, as discussed in WO 2005037955 and US Pub. Appln. 2005/0123760. No mention of the present invention's POSS compositions or uses is discussed in these references. US Pub. Appln. 2005/0090015 discloses composite materials having POSS molecules as a polymer matrix. No mention of the present invention's POSS compositions or uses is discussed.

Another method to improve this detection technique is to make modifications and improvements within the remote sensing system through improved instrumentation and signal processing algorithms. For example, passive Fourier Transform IR spectroscopy (FTIR) has been used for the remote detection of plumes of ethanol vapor at ground level from aircraft flying at 2,000-3,000 feet. This method must overcome constantly changing background radiance and requires a costly and complex process of data collection in order to train the signal processing algorithms. [See Tarumi, T., et al., Appl. Spectroscopy 57(11), 1432-1441 (2003).] In another example, passive FTIR has also been used in the remote detection of bacterial bio-aerosols at a distance of 3 km [Avishi, B. D., Optics Express 11(5), 418-429 (2003)] and methane leaks [Cosofret, B. R. et al., Proceed. of SPIE (International Soc. For Optical Engineering) 5584 (Chem. and Bio. Standoff Detection II) 93-99 (2004)]. These methods also require complex algorithms to address the problem of faint thermal emissions or absorptions superimposed on a fluctuating ambient thermal radiance background.

Lasers play a key role in many remote sensing techniques. One such laser system uses Laser Radar (LADAR) where laser beams scan an object and process the signal echoed from the object. The LADAR processor searches for familiar patterns and compares them to patterns on file. LADAR can identify 15 cm features from a distance of 1 km. Another laser system is differential absorption (DIAL) Lidar (laser detection and ranging) that is used to measure concentrations of species in the atmosphere. DIAL Lidar uses two different wavelengths such that one wavelength is absorbed by the molecule of interest while the other wavelength is not. The difference in intensity between the two return signals can be used to determine the concentration.

For long-range remote sensing, pulsed Lidars are preferable to continuous wave (CW) Lidars. LADAR/Lidar systems generally utilize electromagnetic radiation at optical frequencies.

The general concept of using nanosensors and lasers to interrogate threat clouds was disclosed by a poster at a DARPA meeting in Washington, D.C. on Mar. 2, 2006 by the present assignee of this application, Michigan Molecular Institute. The present invention compositions were not disclosed, only the generalized method.

Clearly, there is an ongoing need to enhance the quality and quantity of information that a remote sensor can obtain from a threat cloud and surfaces to evaluate any contamination.

### BRIEF SUMMARY OF THE INVENTION

This invention modifies a threat cloud or surface to enable it to send higher quality information to a remote sensor. This method uses POSS nanoparticles carrying sensor groups (as “POSS nanosensors”) that change their two- or one-photon fluorescence properties on interaction with a threat cloud or surface, remotely probing the enhanced properties of the cloud or surface using an ultrafast femtosecond infrared laser, and analyzing the resulting fluorescence data.

Specifically, this invention provides POSS nanosensor compounds of the formula

POSS [R]ₙ[(CH₂)ₓZ]₈₋ₙ Formula I

wherein:
each R is C₁-C₁₈ alkyl, including linear or cyclic alkyl, or C₃-C₁₈ aryl, where the R group may be the same or different;
x is 0 or the integer from 1 to 3; and
Z is a fluorophore that has a two-photon cross section of about 10⁻³ GM or above.

### BRIEF DESCRIPTION OF THE DRAWINGS

FIG. 1 illustrates an array of different nanosensors that interact with chemicals in an air flow or cloud. An ultrafast laser causes two-photon fluorescence to occur within the array. The intensity and wavelength of array fluorescence depends upon the content of the air stream as different vapors in the air stream generate different fingerprints. The fluorescence coming from the array is analyzed and when fingerprints associated with explosives or other materials of concern are detected, then a warning is given.

FIG. 2 illustrates that when 2 or more nanosensors were used in an array, fingerprints can be constructed. These fingerprints were constructed using one-photon wavelength shifts (see Table 1 in Example 3) for a range of solvents measured relative to chloroform.

FIG. 3 demonstrates that a four-component array can distinguish G nerve agent simulants (DMMP and DFP), a VX nerve agent simulant (acephate) and a mustard simulant (CEES).

FIG. 4 demonstrates that a four-component array can distinguish water and a homologous series of alcohols, with the exception of ethanol and 1-propanol.

### DETAILED DESCRIPTION OF THE INVENTION

### Glossary

The following terms as used in this application are to be defined as stated below and for these terms, the singular includes the plural.
Acephate means O,S-dimethyl acetylphosphoramidothioate
1-BuOH means 1-butanol
2-BuOH means 2-butanol
CEES means chloroethyl ethyl sulfide
DFP means diisopropylfluorophosphate
DIAL Lidar means differential absorption laser detection and ranging
DMMP means nerve agent simulant dimethyl methylphosphonate
Et₂O means diethyl ether
EtOH means ethanol
FTIR means Fourier Transform infrared spectroscopy
GM means Goppert-Mayer unit (10⁻⁵⁰ cm⁴s/photon)
HFIP means hexafluoroisopropanol (1,1,1,3,3,3-hexafluoropropan-2-ol)
LADAR means Laser Radar
LIF means laser induced fluorescence
MALDI-TOF MS means matrix-assisted laser desorption ionization time of flight mass spectroscopy
MeCN means acetonitrile
MeOH means methanol
NBD means nitrobenzoxadiazole
POSS means polyhedral oligosilsesquioxanes
1-PrOH means 1-propanol
2-PrOH means 2-propanol
PyMPO means [1-(3-succinimdyloxycarbonyl)benzyl]-4-[5-(4-methoxyphenyl)oxazol-2-yl)pyridinium bromide]
RT means room temperature or ambient temperature, from about 20 to about 25° C.
SNAFL means seminaphthofluoresceins
SNARF means seminaphthorhodafluors
TLC means thin layer chromatography
TNT means trinitrotoluene

### Discussion

POSS molecules are stoichiometrically well-defined cage compounds prepared by the hydrolysis and condensation of trifunctional silanes of the form RSiX₃ [see, for example, Scott, D., J. Am. Chem. Soc. 68, 356 (1946) and Varonkov, M. G. et al., Topics Curr, Chem. 102, 199-236 (1982)]. These condensation reactions can generate products ranging from small molecules, oligomers, and clusters to resins of highly complex structure. Which products are obtained from these condensation reactions is highly dependent upon silane and water concentration, pH, temperature, solubility and catalyst [see Brinker, C. J. et al., Sol-Gel Sci.: The Physics and Chemistry of Sol-Gel Processing, Academic Press, San Diego (1990)].

The POSS used in this invention are fully condensed compounds of the form R₈Si₈O₁₂ with a distance of about 1.5 nm between R groups on the adjacent corners of the POSS cage (see Formula II).

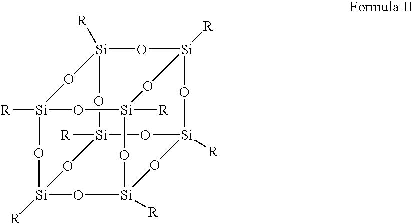

These compounds of Formula II were selected because they are of a precisely defined size, commercially available (Hybrid Plastics, Inc.) with a variety of functional groups. Also they have been used in an extremely wide range of syntheses and applications [see, for example, Feher, F. J., et al., Polyhedron 14, 32-39 (1995); Lichtenhahn, J. D., Polymeric Materials Encyclopedia, ed. Salamone, J. C., CRC Press: New York, 1996, Vol. 10, 7768-7778; and Yang, K. et al., U.S. Pat. No. 0120915 A1]. Thus these compounds are ideal nano-scaffolds for carrying the laser active sensor groups required for the present invention's remote detection technique.

POSS is the smallest possible nano-silica particle that is commercially available and inexpensive. Generally, if groups are placed on the smallest possible unit, then the highest possible density of groups can be achieved (i.e., the greatest number of groups per unit mass of material or per unit volume in a cloud or solution). The higher the density of sensor groups on the nano-material the greater the sensitivity and performance of the sensor system per unit mass of material. This makes the amount of material required less and thus less expensive for this use.

These POSS compounds of Formula II were then modified by attaching fluorophores onto the POSS scaffold to make the desired POSS nanosensors. This technique prevents the self-quenching of the sensor particle that often occurs when the fluorophoric dyes are used on their own, for example when fluorescein interacts with another molecule of fluorescein it ceases to fluoresce. The POSS scaffold keeps the fluorophores separate from each other and thereby enables them to continue emitting light. The present POSS nanosensor compounds of Formula I are the first use of fluorescent sensor groups on POSS. The wavelength-shifting dyes for the Z groups are commercially available and widely used for biomedical fluorescence imaging and microscopy of cells, tissues and organisms. However, the Formula I nanosensor compounds of Formula I have not previously been made for use in an array for any application.

The POSS nanosensors of this invention are shown by the following formula

POSS [R]ₙ[(CH₂)ₓZ]₈₋ₙ Formula I

wherein:
each R is C₁-C₁₈ alkyl, including linear or cyclic alkyl, or C₃-C₁₈ aryl, where the R group may be the same or different;
x is 0 or the integer from 1 to 3; and
Z is a fluorophore that has a two-photon cross section of about 10⁻³ GM or above.

A preferred group of R terms for Formula I are those where one R═C₁-C₃ alkyl and the other seven R groups are identical to one another but differ from the first R group. Particularly preferred are those POSS entities of Formula II that have seven R=isobutyl groups and one R=n-propyl as the linker moiety.

Some specific Z moieties that can be used are shown by the following Formula I structures:

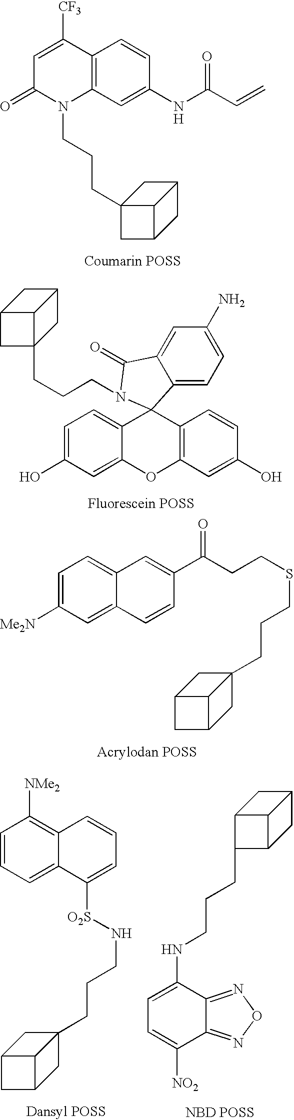

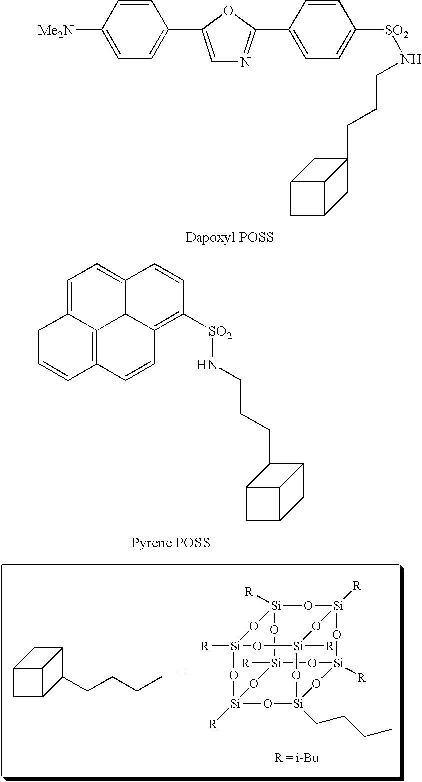

The fluorophores that are attached to POSS and make the POSS nanosensors of this invention include: environment-sensitive fluorophores such as Cascade Yellow; PyMPO; pH sensitive fluorescent labels such as fluoresceins, carboxyfluoresceins, fluorescein diacetates, SNARF, SNAFL, 8-hydroxypyrene-1,3,6-trisulfonic acid, Oregon Green and dichlorofluoresceins; and fluorescent labels sensitive to reactive oxygen species such as trans-1-(2-methoxyvinyl)pyrene, proxyl fluorescamine, lucigenin, nitro blue tetrazolium salts and diphenyl-1-pyrenylphosphine. Especially preferred are coumarin, fluorescein, acrylodan, dansyl, NBD, dapoxyl and pyrene.

The fluorophores or dyes with large two-photon cross sections are preferable, e.g., fluorescein and coumarin have two-photon cross sections of about 100 GM and about 10 GM, respectively. [See Xu, C., et al., J. Opt. Soc. Am. B 13, 481-491 (1996); Lakowicz, J. R., et al., Topics in Fluorescence Spectroscopy Vol. 5, 1997.] Thus preferably the Z groups in Formula I have a two-photon cross section of 10⁻³ GM or above, preferably from about 10⁻³ to about 10⁴ GM. Such material can absorb two photons essentially simultaneously that are equivalent to one photon with the energy of the two added together. [See, for example, Prasad, P. N. et al., Intro. To Nonlinear Optical Effects in Molecules and Polymers, pub. John Wiley and Sons Inc., 1991, NY, pg 178; Denk, W., et al., Science 248, 73-76 (1990).] Although the probability of simultaneous two-photon absorption is very low, such events can occur with greater frequency at the point of focus of a high-power pulse femtosecond laser with pulse duration shorter than 100 fs (10⁻¹³ sec). Such lasers that are ultrafast (picosec to femtosec) are available from various companies such as Spectra Physics, Inc., Coherent, Inc. and Photonic Solutions, Inc.

At this focus point, the photon density is high enough for the fluorophore to absorb two photons simultaneously, but the moderate mean power levels are low enough not to damage the sample or induce excessive photobleaching. Because two-photon excitation of the fluorophore only happens at the focus, it is possible to control the distance at which remote sensing is carried out and to study the different optical sections of a sample. Thus this technique makes this method ideal to analyze threat clouds or to focus on contaminated surfaces from a distance.

Compared to absorption, LIF provides orders of magnitude greater sensitivity. In some cases, LIF has been used for the detection of fluorescent signals from single molecules [Betzig, E., et al., Science 257, 189-195 (1992)]. In contrast, conventional one-photon LIF requires an ultraviolet (UV) laser to excite the reporter molecules to an excited state from which they can emit. Given that the probability of scattering caused by air molecules and particles (e.g., dust, soot, pollen, and salt from the oceans) in the air has an inverse-cubed dependence on the wavelength, the use of UV light is problematic. Also scattered laser light is not coherent and is incapable of inducing two-photon excitation because this nonlinear optical process requires coherent pulsed excitation. Thus, a near infrared femtosecond laser source is vastly preferred and such infrared portion of the spectrum reduces scattering by an order of magnitude. The two-photon LIF provides enhanced background-free imaging even in the presence of scattering particles such as dust, sand and water droplets.

This LIF imaging has already been used in a variety of applications. For example, LIF has been used for functional imaging through biological tissue [Dela Cruz, J. M. et al., Proceed. of the Natl. Acad. of Sci. USA 101(49), 16996-17001 (2004)] and micron resolution was achieved even as the laser transmitted through 1 mm of scattering tissue. LIF has been used for remote detection of TNT where a single laser beam is used to photodissociate TNT and also detect its photofragment—vibrationally excited NO—by LIF. This method worked over a distance of 2.5 m and had a response time of 15 sec. [Heflinger, D. et al., Optics Commun. 204, (1-6), 327-331 (2002)]. LIF has been used for remote identification of minerals [Seigal, H. O., Can. Patent Appln. 2,355,993 (2003)]. Recently, ultrashort terawatt Lidar pulses have been used to remotely detect and identify biological aerosols by introducing two-photon excited fluorescence in riboflavin-containing particles at distances of 2 km [Mejean, G. et al., Appl. Physics B: Lasers and Optics 78(5), 535-537 (2004); Fisch, M. B., et al., SPIE, Paper 4199-05 (2000)].

Presently, most sensing approaches are designed to search for one particular chemical and are not suited for the identification of clouds containing one or more unknowns.

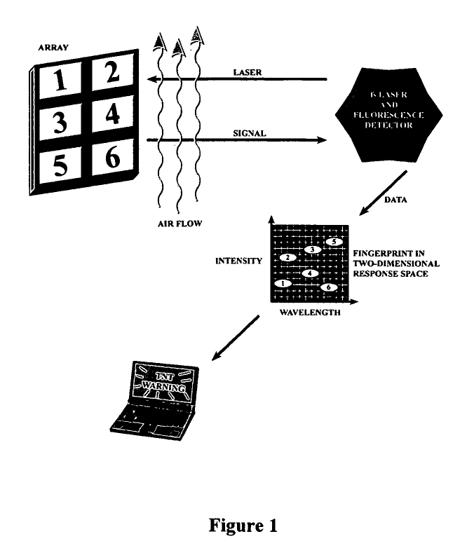

An orthogonal approach is common in other sensor technologies such as arrays of gravimetric surface acoustic wave (SAW) sensors for vapor detection where each sensor in the array is coated with a different polymer [McGill, R. A., et al., CHEMTECH24, 27-37 (1994); Hartmann-Thompson, C., EP Patent Appln. 2004/04256628 and US Pub. Appln. 2005/0090015; Hartmann-Thompson, C. et al., Chem. Mater. 16(24), 5357-5364 (2004)]. This same principle can be applied to probing of threat clouds by introducing a mixture of different POSS nanosensors to a cloud or air stream (See FIG. 1). Each POSS nanosensor will give a different and distinctive response to the constituents of the cloud resulting in a readable fluorescence fingerprint. Thus, the materials you wish to detect will determine the POSS nanosensor mixture used.

Nanosensors such as quantum dots, bioluminescent molecules, nanoporous silicon particles and carbon nanotubes which change their optical characteristics on exposure to analytes such as explosive vapors are known [www.nano-proprietary.com]. These optical changes can be remotely detected over large distances using photomultipliers. However, sensors of this type are normally engineered to detect a particular analyte (e.g., hydrogen in fuel cells, water, glucose, etc.) and are less able to enable detection of a cloud of unknown gases.

In one aspect of the present invention the POSS nanosensors should give a fluorescent signal response as soon as they physically interact with the constituents of the threat cloud. The signal should also persist, since the interaction between the nanosensor and the threat vapor is strong due to solvation and/or hydrogen bonding. The desired POSS nanosensors are introduced into the threat cloud by dispersing the POSS nanosensor material within the threat cloud. It is envisioned that a variety of dispersal devices could be used, such as launching and subsequently exploding a canister of the POSS nanosensors in the cloud, prepositioning canisters of the POSS nanosensors which could be remotely discharged, and launching canisters that disperse the POSS nanosensors as part of a pressurized aerosol mixture. It is anticipated the 37 mm or 40 mm launchers already found in the inventory of many military and police agencies would easily adapt to project canisters of POSS nanosensors. Such a 37 mm or 40 mm canister dispersal method could be used to penetrate a target building's windows at close range or projected to distances on the order of 500 meters. As a variety of 37 mm and 40 mm canisters have been developed to disperse lachrymator agents such as chloroacetophenone, it is anticipated that similar canisters could disperse useful quantities of the POSS nanosensors into a threat cloud. With very little effort, any weapon system that is already capable of dispersing chemical agents could be adapted to disperse POSS nanosensors.

Additionally, it is anticipated that the POSS nanosensors could be dispersed by dropping canisters from the flare or chaff countermeasure dispensers commonly found on military aircraft. It is also envisioned that prepositioned, remotely activated canisters of POSS nanosensors could also be used to disperse the POSS nanosensors into areas likely to become contaminated by chemical attacks or industrial accidents. Additionally, existing cloud seeding or crop dusting techniques could be used to apply the nanosensors to the threat cloud.

In a second aspect of this invention the nanosensors are used to detect whether hazardous chemicals are present on surfaces after a chemical warfare agent attack or an industrial accident/incident. After such an event, the surfaces are cleaned or decontaminated in some manner. After such cleaning, a further need arises to detect whether the decontamination process was effective and if the surface is free of such hazardous materials. The POSS nanosensors of this invention may be used for this purpose in a remote manner. The POSS nanosensors are put into a solution that is applied remotely to the desired surface from a range of containers or canisters that allows the POSS nanosensor solution to be painted, spritzed, sprayed or hosed onto that surface.

In a further aspect of this invention, some of the surfaces that have become contaminated by hazardous chemicals may absorb into the surface material (e.g., low volatility, sticky materials such as the nerve agent VX and blister agents). Such subsurface hazardous species are often not removed by customary surface decontamination processes, but may leach out of the surface later and then pose a threat/danger. The present POSS nanosensors can be used to detect such subsurface hazards.

Another aspect of this invention provides a method for evaluating the constituents of a contaminated airstream, such as a building, airplane, ship, vehicle, HVAC system or a personal respirator, by laser interrogation of an array of Formula I nanosensors on a substrate, with the airstream passing over the substrate. Such a substrate might be incorporated into a standard filter or vent grid.

The POSS nanosensors of Formula 1 are expected to display stability at room temperature when maintained in the dark for significant period of time such that arrays could be made and maintained under these storage conditions for later use. The toxic or hazarodous features of these POSS nanosensors for handling does not presently appear any greater than standard laboratory chemicals.

For the following examples, the various equipment and methods were used to run the various described tests for the results reported in the examples described below.

### Equipment and Methods

The structure of the POSS nanosensors is shown by Formula I. The examples demonstrate that the POSS nanosensors of Formula I change the wavelength of emission by as much as ±60 nm and/or the intensity of light they emit in response to the analyte being detected in a cloud form, on surfaces or in solution in a range of solvents.

### Flash Column Chromatography (TLC)

Flash column chromatography was carried out using a column packed with silica gel (Davisil, grade 633, 200-425 mesh, 60 Å, 99+%) and various fractions were monitored by thin layer chromatography (TLC) using ME Science aluminum-backed silica gel 60 F-254 TLC plates.

### NMR Spectra

¹H and ¹³C NMR spectra were recorded on a Varian Unity 400 MHz NMR spectrometer equipped with a 5 mm multi-nuclei probe. Solvent signals were used as internal standards and chemical shifts are reported relative to tetramethylsilane (TMS).

### MALDI-TOF MS

Electrospray mass spectra were obtained using an Agilent 1100 Series LC-MSD Ion Trap with electrospray ionization. MALDI-TOF mass spectra were measured by M-Scan, Inc. (West Chester, Pa.) using an Applied Biosystems Voyager DE-Pro instrument. A 2,5-dihydroxybenzoic (DHB) acid matrix was used and samples were dissolved in chloroform.

### UV

UV spectra were recorded on a Varian Cary 1E UV/Vis Spectrophotometer. One-dimensional fluorescence spectroscopy was carried out using an Ocean Optics LS-450 gated spectrofluorometer.

### FTIR

IR spectra were recorded on a Nicolet 20DXB FTIR spectrometer and samples were prepared for analysis by solution casting onto potassium bromide discs.

The invention will be further clarified by a consideration of the following examples, which are intended to be purely exemplary of the present invention.

### Example 1

### Preparation of POSS Nanosensors of Formula I where the Wavelength of Emission is Changed in Response to an Analyte

### Part 1a: 1-[6-(Dimethylamino)-2-naphthalenyl][3-[3,5,7,9,11,13,15-heptakis(2-methylpropyl)pentacyclo[9.5.1.1^{3,9}.1^{5,15}.1^{7,13}]octasiloxan-1-yl]propylthio]-1-propanone; Acrylodan POSS

Acrylodan (0.050 g; 0.221 mmol) and thiol POSS (0.197 g; 0.221 mmol) were stirred in chloroform under nitrogen at RT for 8 days and at reflux for a further 6 days. Reaction progress was monitored by TLC. Chloroform was removed in vacuo and the crude product was purified by flash column chromatography (100% chloroform) to give the desired product as a yellow solid (0.18 g; 73% yield), Rf=0.40 (CHCl₃). Its spectra are as follows:

UV (CHCl₃): λₘₐₓ (nm) 366; and

Fluorometer (380 nm lamp, CHCl₃): λₑₘ (nm) 446; and

IR (thin film): ν (cm⁻¹) 2956, 2929, 2905, 2868 (CH₃ and CH₂, sym and asym), 1730 (C═O), 1667, 1616, 1501, 1464, 1379 (NMe₂), 1360, 1327, 1227 (SCH₂ wag), 1105 (SiOSi asym); and

¹H NMR (CDCl₃): δ (ppm) 0.56-0.59 (m; SiCH₂), 0.69-0.77 (t; SiCH₂), 0.93-0.94 (d; CH₃), 1.34-1.43 (m; SiCH₂CH₂), 1.78-1.88 (m; CH), 2.57-2.61 (t; SCH₂), 2.90-2.94 (t; SCH₂), 3.09 (s; N(CH₃)₂), 3.30-3.34 (t; CH₂C═O), 6.86 (d; ArH ortho to N(CH₃)₂), 7.14-7.17 (dd; ArH ortho to N(CH₃)₂), 7.61-7.63 (d; ArH meta to N(CH₃)₂), 7.77-7.79 (d; ArH meta to C═O), 7.88-7.91 (dd; ArH ortho to C═O), 8.30 (d; ArH ortho to C═O); and

¹³C NMR (CDCl₃): δ (ppm) 11.0 (SiCH₂), 14.1 (CH2), 22.5 (i-BuC) 23.0 (SCH₂), 23.8 (i-BuC), 25.7 (i-BuC), 28.9 (SCH₂), 30.4 (CC═O), 38.8 (N(CH₃)₂), 116.3 (ArCH), 124.5 (ArCH), 126.3 (ArCH), 128.8 (ArCH), 130.9 (ArCH), 132.5 (ArCH); and

MS (EI positive mode): m/z 1139 (Calc. 1140, molecular ion plus sodium).

### Part 1b: 5-(Dimethylamino)-N-[3-[3,5,7,9,11,13,15-heptakis(2-methylpropyl)pentacyclo[9.5.1.1^{3,9}.1^{5,15}.1^{7,13}]-octasiloxan-1-yl]propyl]-1-naphthalenesulfonamide; 1,5-Dansyl POSS

1-Dimethylaminonaphthalene-5-sulfonyl chloride (0.200 g; 0.741 mmol) and amino POSS (0.648 g; 0.741 mmol) were stirred in chloroform under nitrogen at RT for 12 days and at reflux for a further 14 days. Reaction progress was monitored by TLC. Chloroform was removed in vacuo and the crude product was purified by flash column chromatography (100% CHCl₃) to give the desired product as an off-white solid (0.57 g; 69% yield), Rf=0.55 (CHCl₃). Its spectra are as follows:

UV (CHCl₃): λₘₐₓ (nm) 341; and

Fluorometer (380 nm lamp, CHCl₃): λₑₘ (nm) 497; and

IR (thin film): ν (cm⁻¹) 2955, 2920,2897, 2866 (CH₃ and CH₂, sym and asym), 1612, 1586, 1576, 1464, 1423, 1407, 1395, 1382, 1363, 1353, 1317(SO₂asym), 1259, 1227, 1186 (SO₂ sym), 1111 (SiOSi asym) ; and

¹H NMR (CDCl₃): δ (ppm) 0.46-0.50 (d; SiCH₂), 0.55-0.59 (t; SiCH₂), 0.91-0.95 (m; CH₃), 1.51-1.53 (m; SiCH₂CH₂), 1.77-1.87 (m; CH), 2.84-2.88 (t; CH₂NHSO₂), 2.89 (s; N(CH₃)₂), 7.18-7.20 (d; ArH para to N(CH₃)₂), 7.50-7.58 (2dd; ArH meta to SO₂ and ArH ortho to N(CH₃)₂), 8.23-8.25 (dd; ArH meta to N(CH₃)₂), 8.27-8.29 (d; ArH para to SO₂), 8.53-8.56 (d; ArH ortho to SO₂); and

¹³C NMR (CDCl₃): δ (ppm) 9.1 (SiCH₂), 22.4 (i-BuC) 23.3 (CH₂) 23.8 (i-BuC), 25.7 (i-BuC), 45.1 (CH₂NHSO₂), 45.7 (N(CH₃)₂), 103.2 (ArC), 114.0 (ArC), 114.9 (ArC), 115.3 (ArC), 118.6 (ArC), 122.9 (ArC), 127.9 (ArC), 129.7 (ArC), 130.2 (ArC), 130.5 (ArC); and

MS (EI positive mode): m/z 1131 (Calc. 1131, molecular ion plus sodium).

### Part 1c: 6-(Dimethylamino)-N-13-[3,5,7,9,11,13,15-heptakis(2-methylpropyl)pentacyclo[9.5.1.1^{3,9}.1^{5,15}.1^{7,13}]-octasiloxan-1-yl]propyl]-2-naphthalenesulfonamide; 2,6-Dansyl POSS

2-Dimethylaminonaphthalene-6-sulfonyl chloride (0.30 g; 1.11 mmol) and amino POSS (0.971 g; 1.11 mmol) were stirred in chloroform (5 mL) under nitrogen at RT for 14 days and then heated at 50° C. for 7 days. Reaction progress was monitored by TLC. Chloroform was removed in vacuo and the crude product was purified by flash column chromatography (100% chloroform) to give the desired product as a pale orange solid (0.83 g; 67% yield). Rf=0.25 (CHCl₃). Its spectra are as follows:

UV (CHCl₃): λₘₐₓ (nm) 324; and

Fluorometer (380 nm lamp, CHCl₃): λₑₘ (nm) 427; and

IR (thin film): ν (cm⁻¹) 3249 (NHSO₂), 2955, 2924, 2896, 2863 (CH₃ and CH₂, sym and asym), 1624, 1510, 1465, 1384 (ArNMe₂), 1330 (SO₂), 1230 (SiCH₂), 1109 (SiOSi asym) 835 (SiOSi sym); and

¹H NMR (CDCl₃): δ (ppm) 0.52-0.55 (d; SiCH₂), 0.56-0.60 (t; SiCH₂), 0.92-0.95 (dd; CH₃), 1.57 (m; CH₂), 1.80-1.88 (m; CH), 2.92-2.97 (m; CH₂NHSO₂), 3.12 (s; N(CH₃)₂), 6.96-7.00 (m; ArH), 7.23-7.25 (d; ArH), 7.67-7.69 (dd; ArH), 7.70-7.72 (d; ArH), 7.79-7.82 (d; ArH), 8.25 (d; ArH); and

¹³C NMR (CDCl₃): δ (ppm) 9.21 (SiCH₂), 22.5, 23.8, 25.7 (BuC), 23.3 (CH₂), 41.5 (CH₂NHSO₂), 45.6 (N(CH₃)₂), 117.3 (ArCH), 123.1 (ArCH), 127.4 (ArCH), 127.6 (ArCH), 128.4 (ArCH), 130.6 (ArCH); and

MS (MALDI-TOF): m/z 1107 (Calc. 1108).

### Part 1d: 5-(Dimethylamino)N-[3-[3,5,7,9,11,13,15-heptakis(2-methylpropyl)pentacyclo[9.5.1.1^{3,9}.1^{5,15}.1^{7,13}]-octasiloxan-1-yl]propyl]-2-naphthalenesulfonamide; 2,5-Dansyl POSS

2-Dimethylaminonaphthalene-5-sulfonyl chloride (0.30 g; 1.11 mmol) and amino POSS (0.971 g; 1.11 mmol) were stirred in chloroform (5 mL) under nitrogen at RT for 7 days and then heated at 50° C. for 9 days. Reaction progress was monitored by TLC. Chloroform was removed in vacuo and the crude product was purified by flash column chromatography (2:1 v/v chloroform-hexane) to give the desired product as a pale yellow solid (1.09 g; 89 % yield). Rf=0.10 (CHCl₃). Its spectra are as follows:

UV (CHCl₃): λₘₐₓ (nm) 407; and

Fluorometer (380 nm lamp, CHCl₃): λₑₘ (nm) 452; and

IR (thin film): ν (cm⁻¹) 3290 (NHSO₂), 2954, 2926, 2900, 2866 (CH₃ and CH₂, sym and asym), 1621, 1593, 1514, 1465, 1367 (ArNMe₂) 1327 (SO₂), 1230 (SiCH₂), 1202, 1109 (SiOSi asym), 830 (SiOSi sym); and

¹H NMR (CDCl₃): δ (ppm) 0.47-0.51 (t; SiCH₂), 0.56-0.60 (t; SiCH₂), 0.91-0.96 (m; CH₃), 1.50-1.56 (m; CH₂), 1.78-1.88 (m; CH), 2.84-2.89 (m; CH₂N), 3.09 (s; N(CH₃)₂), 7.03 (m; ArH), 7.28-7.31 (dd; ArH), 7.37-7.41 (dd; ArH), 7.84-7.86 (d; ArH), 7.92-7.94 (d; ArH), 8.50-8.52 (d; ArH); and

¹³C NMR (CDCl₃): δ (ppm) 9.2 (SiCH₂), 22.4, 23.8, 25.7 (BuC), 23.3 (CH₂), 40.8 (NCH₃), 45.7 (SO₂NCH₂), 117.9 (ArCH), 124.5 (ArCH), 125.2 (ArCH), 132.5 (ArCH), 134.3 (ArCH), 136.2 (ArCH); and

MS (MALDI-TOF): m/z 1107 (Calc. 1108).

### Part 1e: N-[3-[3,5,7,9,11,13,15-Heptakis(2-methylpropyl)pentacyclo[9.5.1.1^{3,9}.1^{5,15}.1^{7,13}]-octasiloxan-1-yl]-propyl]-1-pyrenesulfonamide; 1-Pyrene POSS

1-Pyrene sulfonyl chloride (0.400 g; 1.32 mmol) and amino POSS (1.160 g; 1.32 mmol) were stirred in chloroform under nitrogen at RT for 14 days. Reaction progress was monitored by TLC. Chloroform was removed in vacuo and the crude product was purified by flash column chromatography (100% chloroform gradient to 1:1 v/v chloroform-hexane) to give the desired product as a pale yellow solid (0.80 g; 53% yield). Rf=0.45 (CHCl₃). Its spectra are as follows:

UV (CHCl₃): λₘₐₓ (nm) 356; and

Fluorometer (380 nm lamp, CHCl₃): λₑₘ (nm) 385 and 399; and

IR (thin film): ν (cm⁻¹) 3288 (NH), 2954, 2915, 2868, 2847 (CH₃ and CH₂, sym and asym), 1456, 1327 (SO₂ asym), 1226, 1193, 1148 (SO₂ sym), 1100 (SiOSi asym), 1033, 845, 744; and

¹H NMR (CDCl₃): δ (ppm) 0.41-0.47 (t; SiCH₂), 0.54-0.56 (t; SiCH₂), 0.84-0.94 (m; CH₃), 1.52-1.54 (m; SiCH₂CH₂), 1.70-1.85 (m; CH), 2.88-2.90 (t; CH₂NHSO₂), 8.08-8.12 (m; ArH), 8.20-8.23 (2d; ArH), 8.29-8.33 (m; ArH), 8.67-8.69 (d; ArH), 8.95-8.97 (d; ArH).

¹³C NMR (CDCl₃): δ (ppm) 9.1 (SiCH₂), 22.4 (i-BuC), 23.3 (CH₂), 23.8 (i-BuC), 25.7 (i-BuC), 45.6 (CH₂NHSO₂), 123.0 (ArC), 123.8 (ArC), 124.1 (ArC), 125.3 (ArC), 126.8 (ArC), 126.9 (ArC), 127.0 (ArC), 127.1 (ArC), 127.5 (ArC), 128.0 (ArC), 130.1 (ArC), 130.2 (ArC), 131.0 (ArC), 131.2 (ArC), 134.9 (ArC); and

MS (EI negative mode): m/z 1137 (Calc. 1139, molecular ion).

### Part 1f: N-[3-[3,5,7,9,11,13,15-Heptakis(2-methylpropyl)pentacyclo[9.5.1.1^{3,9}.1^{5,15}.1^{7,13}-octasiloxan-1-yl]-propyl]-7-nitro-2,1,3-benzoxadiazol-4-amine; NBD POSS

4-Chloro-7-nitrobenzfurazan (NBD chloride, 0.401 g; 2.00 mmol) and amino POSS (1.758 g; 2.00 mmol) were stirred in chloroform under nitrogen at RT for 14 days. Reaction progress was monitored by TLC. Chloroform was removed in vacuo and the crude product was purified by flash column chromatography (100% chloroform gradient to 1:1 v/v chloroform-hexane) to give the desired product as an orange solid (1.10 g; 53% yield). Rf=0.35 (CHCl₃). Its spectra are as follows:

UV (CHCl₃): λₘₐₓ (nm) 446; and

Fluorometer (470 nm lamp, CHCl₃): λₑₘ (nm) 523; and

IR (thin film): ν (cm⁻¹) 3250 (NH), 2955, 2912, 2870, 2849 (CH₃ and CH₂, sym and asym), 1574 (NO₂ asym), 1380, 1305 (NO₂ sym), 1288, 1254, 1094 (SiOSi asym); and

¹H NMR (CDCl₃): δ (ppm) 0.60-0.62 (d; SiCH₂), 0.74-0.78 (t; SiCH₂), 0.94-0.96 (m; CH₃), 1.80-1.90 (m; CH), 1.90-1.92 (m; SiCH₂CH₂), 3.50-3.51 (t; CH₂NHAr), 6.16-6.18 (d; ArH meta to NO₂), 8.48-8.50 (d; ArH ortho to NO₂); and

¹³C NMR (CDCl₃): δ (ppm) 9.3 (SiCH₂), 22.1 (CH₂), 22.4 (i-BuC), 23.8 (i-BuC), 25.7 (i-BuC), 48.9 (NHCH₂), 134.7 (ArC), 136.3 (ArC), 140.7 (ArC), 143.2 (ArC), 143.9 (ArC), 144.3 (ArC); and

MS (EI positive mode): m/z 1039 (Calc. 1038, molecular ion plus sodium).

### Part 1g: 4-[5-[4-(Dimethylamino)phenyl]-2-oxazolyl]-N-[3-[3,5,7,9,11,13,15-heptakis(2-methylpropyl)pentacyclo[9.5.1.1^{3,9}.1^{5,15}.1^{7,13}]-octasiloxan-1-yl]propyl]benzenesulfonamide; Dapoxyl POSS

Dapoxyl chloride (0.040 g; 0.110 mmol) and amino POSS (0.096 g; 0.110 mmol) were stirred in chloroform under nitrogen at RT for 14 days. Reaction progress was monitored by TLC. Chloroform was removed in vacuo and the crude product was purified by flash column chromatography (1:1 v/v chloroform-hexane gradient to 100% chloroform) to give the desired product as a deep yellow solid (0.12 g; 91% yield). Rf=0.10 (CHCl₃). Its spectra are as follows:

UV (CHCl₃): λₘₐₓ (nm) 323; and

Fluorometer (380 nm lamp, CHCl₃): λₑₘ (nm) 500; and

IR (thin film): ν (cm⁻¹) 3300 (NH), 2964, 2913, 2885, 2846 (CH₃ and CH₂, sym and asym), 1506, 1461, 1330 (SO₂ asym), 1259, 1225, 1150 (SO₂ sym), 1099 (SiOSi asym), 1015, 801, 740; and

¹H NMR (CDCl₃): δ (ppm) 0.58-0.62 (m; SiCH₂), 0.93-0.97 (m; CH₃), 1.60 (m; CH₂), 1.81-1.86 (m; CH), 2.84 (t; CH₂NH), 3.03 (s; N(CH₃)₂); 6.74-6.78 (d; ArH), 7.30 (s; ArH), 7.59-7.61 (d; ArH), 7.93-7.95 (d; ArH), 8.19-8.21 (d; ArH); and

¹³C NMR (CDCl₃): δ (ppm) 9.6 (SiCH₂), 22.7 (i-BuC) 23.2 (CH₂), 24.1 (i-BuC), 25.9 (i-BuC), 40.5 (N(CH₃)₂), 45.8 (CH₂NHSO₂), 112.3 (ArCH), 115.7 (ArC), 121.4 (ArCH), 125.9 (ArCH), 126.5 (ArCH), 127.7 (ArCH), 131.6 (ArC), 140.9 (Arc), 150.9 (oxazole ArC), 153.5 (oxazole ArC), 158.3 (oxazole ArC); and

MALDI-TOF MS: m/z 1202 (Calc. 1201); and

MS (EI positive mode): m/z 1081 (Calc. 1081, M⁺ minus PhN(CH₃)₂ fragment).

### Part 1h: 1-[[3-[[[3-[3,5,7,9,11,13,15-Heptakis(2-methylpropyl)pentacyclo[9.5.1.1^{3,9}.1^{5,15}.1^{7,13}]-octasiloxan-1-yl]propyl]amino]carbonyl]phenyl]methyl]-4-[5(4-methoxy-3-sulfophenyl)-2-oxazolyl]pyridinium, inner salt; Cascade Yellow POSS

Cascade Yellow succinimide ester (30 mg; 0.053 mmol) and amino POSS (0.0456 g; 0.053 mmol) were stirred in chloroform (5 mL) under nitrogen at RT for 6 days. Reaction progress was monitored by TLC. Chloroform was removed in vacuo and the crude product was purified by flash column chromatography (100% chloroform gradient to 3:1 v/v chloroform-methanol) to give the desired product as a bright yellow powder solid (30 mg; 42% yield). Rf=0.25 (CHCl₃). Spectra are as follows:

UV (CHCl₃): λₘₐₓ (nm) 438; and

Fluorometer (380 nm lamp, CHCl₃): λₑₘ (nm) 529; and

MS (EI positive mode): m/z 1316 (Calc. 1323, molecular ion).

### Example 2

### POSS Nanosensors of Formula I where the Intensity of the Light Emitted is Changed in Response to an Analyte

### Part 2a: Fluorescein functionalized POSS

Fluoresceinamine isomer 1 (0.218 g; 0.628 mmol) and amino POSS (0.549 g; 0.628 mmol) were dissolved in a mixture of methanol (2 mL) and chloroform (2 mL) and stirred for 7 days at 60° C. The solvents were removed in vacuo and the crude product was purified by flash column chromatography (1:9 v/v methanol-chloroform, gradient to 1:1 v/v methanol-chloroform). Fluorescein functionalized POSS lactam was obtained as a deep orange solid (0.20 g; 26%); Rf=0 (1:9 v/v methanol-chloroform). Its spectra are as follows:

UV (1:1 v/v MeOH—CHCl₃): λₘₐₓ (nm) 458 and 482; and

Fluorometer (470nm lamp): λₑₘ (nm) 535; and

IR (thin film): ν (cm⁻¹) 3319 (OH), 3207 (lactam NH), 2956, 2929, 2900, 2859 (sym, asym CH₂ and CH₃), 1710 (lactam C═O) 1627, 1597, 1497, 1464 (iBu), 1383 (iBu), 1324, 1272, 1224, 1206, 1172, 1102 (asym SiOSi), 1036, 836 (sym SiOSi); and

¹H NMR (1:1 v/v CDCl₃—CD₃OD): δ (ppm) 0.61-0.65 (m; SiCH₂), 0.98-1.00 (d; CH₃), 1.17-1.22 (t; CH₂), 1.84-1.93 (m; CH), 6.59-6.63-(dd; ArH ortho to OH), 6.66-6.68 (d; ArH ortho to OH), 6.89-6.93 (d; ArH ortho to C═O), 6.95-6.98 (dd; ArH para to C═O), 7.03-7.07 (d; ArH meta to OH), 7.26-7.28 (d; ArH meta to C═O); and

¹³C NMR (1:1 v/v CDCl₃—CD₃OD): δ (ppm) 8.01 (SiCH₂), 20.4 (CH₂), 21.3, 23.0, 24.1 (iBu), 40.9 (NCH₂), 101.7, 111.3, 112.5, 116.1, 117.5, 127.1, 129.8, 134.5, 148.7, 155.1, 161.2, 167.9, 171.5; and

MALDI-TOF MS (DHB): m/z 1207 (Calc. 1204).

### Part 2b: Coumarin functionalized POSS

7-(4-Trifluoromethylcoumarin)acrylamide (0.426 g; 1.50 mmol) and amino POSS (1.32 g; 1.50 mmol) were stirred in 1-methyl-2-pyrollidone (NMP) for 5 days at room temperature and a further 6 days at 90° C., and monitored by thin layer chromatography. NMP was removed by vacuum distillation and the crude product was purified by flash column chromatography (100% CHCl₃). Coumarin functionalized POSS lactam was obtained as a yellow solid (0.35 g, 20%); Rf=0.10 (CHCl₃). Its spectra are as follows:

UV (CHCl₃): λₘₐₓ (nm) 342; and

Fluorometer (380 nm lamp): λₑₘ (nm) 488 and 445; and

IR (thin film): ν (cm⁻¹) 3252 (NH lactam), 3081 (C═CH), 2956, 2933, 2900, 2867 (sym, asym CH₂ and CH₃), 1693 (broad, C═O acrylamide and lactam), 1612, 1582, 1523, 1505, 1461 (iBu), 1427, 1401, 1331 (iBu), 1298, 1279, 1117 (asym SiOSi), 1028, 836 (sym SiOSi); and

¹H NMR (CDCl₃): δ (ppm) 0.32-0.45 (m; SiCH₂), 0.73-0.77 (m; CH₃), 1.60-1.70 (m; CH), 4.69-4.71 (d; C═CH₂), 5.54-5.57 (t; CH═CH₂), 6.25-6.27 (d; C═CH₂), 6.44 (s; CF₃C═CH), 7.39-7.43 (dd; ArH), 7.50-7.54 (dd; ArH), 7.80-7.81 (d; ArH ortho to acrylamide); and

¹³C NMR (100% CDCl₃): δ (ppm) 18.8 (SiCH₂), 23.3, 22.0, 25.3, 29.0 (iBu and CH₂CH₂Si), 61.3 (CH₂N), 90.5 (CF₃), 107.0 (ArCH), 108.4 (ArC), 112.7 (ArCH), 116.1 (ArCH), 119.3 (ArC), 123.0 (ArC), 125.0 (ArCH), 127.7 (C═CH), 130.7 (C═CH2), 143.5 (ArCNH), 164.1 (NHC═O), 176.9 (C═O lactam); and

MALDI-TOF MS (DHB): m/z 1170 (Calc. 1140).

### Example 3

One-photon fluorescence λₑₘ (nm) responses of POSS nanosensors of Formula I in solution (˜1×10⁻³ M) with a range of solvents, see Table 1 below. This Table 1 shows fluorescence data (absolute and relative to chloroform) for POSS nanosensor compounds exposed to various analytes. *λₑₓ=380 nm in all experiments except for NBD POSS 7 where λₑₓ=470 nm.

TABLE 1

| | Acrylodan | | 1,5-Dansyl | | 2,6-Dansyl | | 2,5-Dansyl | | NBD | | Dapoxyl | |
|---|---|---|---|---|---|---|---|---|---|---|---|---|
| | POSS 1 | | POSS 2 | | POSS 3 | | POSS 4 | | POSS 7* | | POSS 8 | |
| | λₑₘ (nm) | | λₑₘ (nm) | | λₑₘ(nm) | | λₑₘ(nm) | | λₑₘ (nm) | | λₑₘ (nm) | |
| CHCl₃ | 446 | 0 | 497 | 0 | 427 | 0 | 452 | 0 | 523 | 0 | 500 | 0 |
| Hexane | 421 | −25 | 448 | −49 | 414 | −13 | 429 | −23 | 504 | −19 | 437 | −63 |
| Toluene | 437 | −9 | 496 | −1 | 425 | −2 | 444 | −8 | 514 | −9 | 492 | −8 |
| Xylene | 437 | −9 | 496 | −1 | 427 | 0 | 443 | −9 | 519 | −4 | 492 | −8 |
| Acetone | 451 | +5 | 492 | −5 | 439 | +12 | 490 | +38 | 525 | +2 | 542 | +42 |
| MeOH | 509 | +63 | 523 | +26 | 437 | +10 | 493 | +41 | 536 | +13 | 554 | +54 |
| EtOH | 502 | +56 | 509 | +12 | 439 | +12 | 490 | +38 | 533 | +10 | 534 | +34 |
| Water | 490 | +44 | 494 | −3 | Insoluble | | Insoluble | | 542 | +19 | 517 | +17 |
| MeCN | 487 | +41 | 514 | +17 | 441 | +14 | 492 | +40 | 530 | +7 | 555 | +55 |
| CEES | 451 | +5 | 496 | −1 | 433 | +6 | 483 | +31 | 524 | +1 | 510 | +10 |
| Acephate | 498 | +52 | 533 | +36 | — | | — | | 549 | +26 | 543 | +43 |
| DFP | 470 | +24 | 495 | −2 | 438 | +11 | 490 | +38 | 498 | −25 | 558 | +58 |
| DMMP | 489 | +43 | 502 | +5 | 441 | +14 | 492 | +40 | 534 | +11 | 503 | +3 |
| 1-PrOH | 500 | +54 | 508 | +11 | 437 | +10 | 489 | +37 | 532 | +9 | 531 | +31 |
| 2-PrOH | 496 | +50 | 514 | +17 | 437 | +10 | 490 | +38 | 532 | +9 | 541 | +41 |
| 1-BuOH | 498 | +52 | 514 | +17 | 436 | +9 | 489 | +37 | 533 | +10 | 535 | +35 |
| 2-BuOH | 494 | +48 | 511 | +14 | 437 | +10 | 488 | +36 | 531 | +8 | 531 | +31 |
| Et₂O | 430 | −16 | 495 | −2 | 425 | −2 | 443 | −9 | 512 | −11 | 495 | −5 |
| HFIP | 536 | +90 | 500 | +3 | 447 | +20 | 504 | +52 | 546 | +23 | 539 | +39 |

From the data in Table 1, it can be concluded that each POSS nanosensor material has a significantly different wavelength of emission in response to each of a chemically diverse range of analytes. Hence a selection from this set of nanosensors is ideal for use in an array to generate unique fingerprints for each of these analytes.

### Example 4

### Arrays of Various POSS Agents

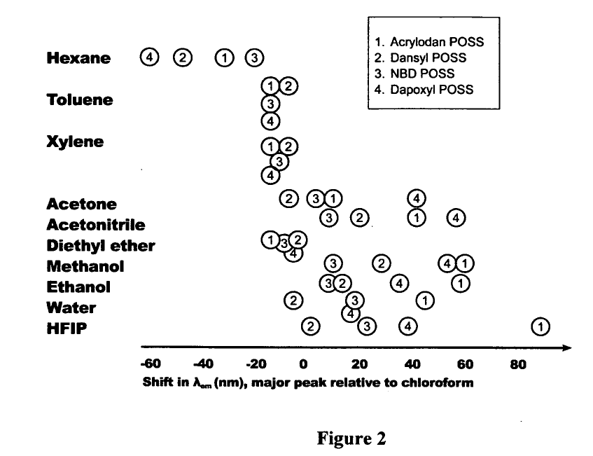

Part 4a: When 3 or more nanosensors were used in an array, fingerprints for common organics and toxic industrial chemicals (TICs) could be constructed as shown in FIG. 2. These fingerprints were constructed using one-photon wavelength shifts (see Table 1 above) measured relative to chloroform.

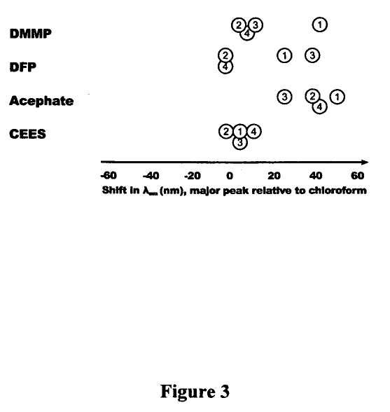

Part 4b: The array was tested with four chemical warfare agent simulants (FIG. 3): dimethyl methylphosphonate and diisopropylfluorophosphate (DMMP and DFP, simulants of G series nerve agents such as soman GD, tabun GA, and sarin GB), O,S-dimethyl acetylphosphoramidothioate (acephate, a simulant of the V series nerve agent VX), and chloroethyl ethyl sulfide (CEES, a simulant of mustard gas HD). The array had good selectivity for all four simulants, and it was particularly notable that the three phosphonate nerve simulants gave markedly different fingerprints, since distinguishing G agents from VX is an on-going challenge in the field of nerve agent detection.

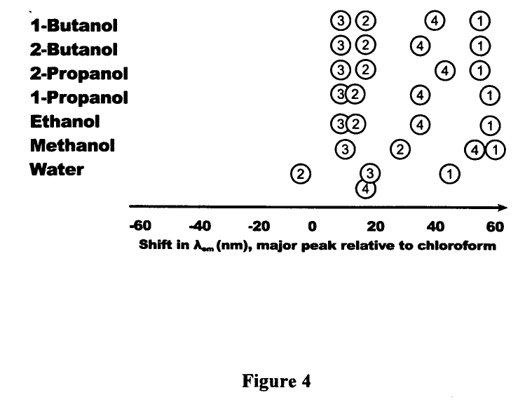

Part 4c: The array was used to study alcohols in greater detail in the third set of experiments (FIG. 4). In this case, the array could distinguish a homologous series of alcohol isomers up to 1- and 2-butanol, with the exception of the ethanol and 1-propanol pair, which gave identical fingerprints.

### Example 5

Solutions of POSS nanosensors (1×10⁻³ M in chloroform or methanol) are stable in the presence of the femtosecond IR laser and continue to give strong two-photon fluorescence after 20 minutes. Laser experiments were carried out using a Spectra Physics Spitfire titanium sapphire regeneratively amplified laser system. The output was centered at 800 nm, the pulse duration was 33 fs, the bandwidth was 29 nm, and the repetition rate was 1 kHz.

### Example 6

POSS nanosensors have good shelf life and give good fluorescence a year after synthesis and are best kept in the dark for maximum useful life. This enables the POSS nanosensors to be made ahead and kept to form an array of desired nanosensors that can be selected for use based on the possible threat constituents.

### Example 7

### Cloud Chamber with Two-Photon Fluorescence

Part 7a: Cloud particles generated in a laboratory cloud chamber persist and give two-photon fluorescence for 5-10 min. A Badger 150 airbrush connected to a cylinder of ultra-high purity nitrogen with the outlet pressure set to 20 psi was used to generate spray clouds in a 5 L covered beaker. One airbrush was charged with 1×10⁻³ M solutions of POSS nanosensors in chloroform or methanol, and a second air brush was charged with DMMP. When DMMP was added to a cloud of NBD POSS, a shift in fluorescence wavelength from 523 nm (yellow green by eye) to 531 nm (yellow by eye) was observed. Some optical sectioning could also be observed, i.e., the laser beam was localized over an approximately one-inch distance in the center of the chamber, and very faint in the remainder of its path through the beaker. Hence a small area in the center of the cloud was effectively being interrogated and emitting most of the light signal detected by the fluorometer.

Part 7b: Cloud experiments as per Example 7a were repeated except that two-dimensional fluorescent optical sections of clouds were studied instead of one-dimensional ˜1 inch laser lines. Optical sections were generated using a laser scanner. A fluorescent optical section of a cloud of NBD POSS responded to the presence of DMMP by giving an 8 nm yellow-green (523 nm) to yellow (531 nm) wavelength shift. A fluorescent optical section of a cloud of dapoxyl POSS responded to the presence of DMMP by giving a 25 nm blue-green (500 nm) to yellow-green (525 nm) wavelength shift.

### Example 8

Two-photon fluorescence data for DMMP surface contamination on anodized aluminum foil, see Table 2 below.

TABLE 2

| | | Response for chloroform |
|---|---|---|
| | Response | nanosensor |
| | for chloroform nanosensor | solution spritzed |
| POSS | solution spritzed | onto DMMP contaminated |
| nanosensor | onto clean surface (control) | surface |
| Dansyl POSS | 497 nm | 500 nm |
| | Blue-green | Blue-green |
| Dapoxyl POSS | 500 nm | 525 nm |
| | Blue-green | Yellow-green |
| NBD POSS | 523 nm | 531 nm |
| | Yellow-green | Yellow |

From the data in Table 2, it can be seen that POSS nanosensors are capable of shifting their wavelengths of emission in response to the nerve agent simulant DMMP when used on a contaminated surface. The same wavelength shifts are seen as in the solution experiment results given in Table 1. Hence, the nanosensor array can generate the same fingerprint for the same analyte either in solution or on a contaminated surface.

Although the invention has been described with reference to its preferred embodiments, those of ordinary skill in the art may, upon reading and understanding this disclosure, appreciate changes and modifications which may be made which do not depart from the scope and spirit of the invention as described above or claimed hereafter.

What is claimed is:

1. POSS nanosensor compounds of the formula
POSS [R]ₙ[(CH₂)ₓZ]₈₋ₙ Formula I
wherein:
each R is C₁-C₁₈ alkyl, including linear or cyclic alkyl, or C₃-C₁₈ aryl, where the R group may be the same or different;
x is 0 or the integer from 1 to 3; and
Z is a fluorophore that has a two-photon cross section of about 10⁻³
GM or above;
provided that n=0 or 1-7 and when n=7, then more than one compound of Formula I is present as an array where the Z fluorophore is different.

2. The compounds of claim 1 where Z is an environment-sensitive fluorophore selected from (a) acrylodan, dansyl, pyrene, NBD, dapoxyl, Cascade Yellow, anthracene or PyMPO; (b) a pH sensitive fluorescent label; and (c) fluorescent labels sensitive to reactive oxygen species.

3. The compounds of claim 2 where Z is a pH sensitive fluorescent label selected from the group consisting of fluorescein, carboxyfluorescein, fluorescein diacetate, SNARF, SNAFL, 8-hydroxypyrene-1,3,6-trisulfonic acid, Oregon Green and dichlorofluorescein.

4. The compounds of claim 2 where Z is a fluorescent label sensitive to reactive oxygen species selected from the group consisting of trans-1-(2-methoxyvinyl)pyrene, proxyl fluorescamine, lucigenin, nitro blue tetrazolium salts and diphenyl-1-pyrenylphosphine.

5. The compounds of claim 1 where Z is coumarin, fluorescein, acrylodan, dansyl, NBD, dapoxyl or pyrene.

6. The compounds of claim 1 wherein Z is a fluorophore that has a two-photon cross section from about 10⁻³ to about 10⁴ GM.

7. A method of evaluating the constituents in a threat cloud remotely by providing the POSS nanosensors of Formula I as defined in claim 1 by launching and dispersing a canister of the POSS nanosensors material within the threat cloud or seeding the threat cloud where the POSS nanosensors change their two- or one-photon fluorescence properties on interaction with a threat cloud, remotely probing the enhanced properties of the cloud using an infrared laser, and analyzing the resulting data.

8. A method of evaluating the contamination of a surface either near by or remotely by applying a solution of POSS nanosensors of Formula I as defined in claim 1 to the desired surface from a range of containers or canisters that allows the POSS nanosensor solution to be painted, spritzed, sprayed or hosed to that surface that change their two- or one-photon fluorescence properties on interaction with a surface, remotely probing the enhanced properties of the surface using an infrared laser, and analyzing the resulting data.

9. The method of claim 7 or 8 wherein the infrared laser is a ultrafast fentosecond laser.

10. A method for evaluating the constituents of a contaminated airstream by laser interrogation of an array of POSS nanosensors of Formula I as defined in claim 1 on a substrate with the airstream passing over the substrate.

11. The method of claim 10 where the airstream is in a building, airplane, ship, vehicle, HVAC system, or a personal respirator.
